(19) United States
(12) Patent Application Publication
(10) Pub. No.: US 2019/0192658 A1
(43) Pub. Date: Jun. 27, 2019

(54) TOPICAL PAIN RELIEF COMPOSITION

(71) Applicant: Melissa Ward, Oklahoma City, OK (US)

(72) Inventor: Melissa Ward, Oklahoma City, OK (US)

(21) Appl. No.: 16/234,380

(22) Filed: Dec. 27, 2018

Related U.S. Application Data
(60) Provisional application No. 62/610,899, filed on Dec. 27, 2017.

(51) Int. Cl. A61K 45/06; A61K 31/05; A61K 31/047; A61K 31/045; A61K 36/899; A61K 36/53; A61K 36/28; A61K 31/355; A61K 47/32; A61K 36/886; A61K 31/10; A61K 31/133; A61K 36/76; A61K 36/81; A61K 36/31; A61P 29/00

(52) U.S. Cl. CPC A61K 45/06; A61P 29/00; A61K 31/047; A61K 31/045; A61K 36/899; A61K 36/53; A61K 36/28; A61K 31/355; A61K 47/32; A61K 36/886; A61K 31/10; A61K 31/133; A61K 36/76; A61K 36/81; A61K 36/31; A61K 31/05

(57) ABSTRACT

A topical pain relief composition including a topical cream that may be rubbed on virtually any area in need of pain relief. It offers an analgesic formulated for relieving pain in the joints or muscles. The composition includes a matrix including a water soluble active ingredient ranging from 20.7% to 25.2% total weight of the matrix, a water insoluble active ingredient ranging from 18.7% to 22.9% total weight of the matrix, and purified water ranging from 56% to 68.8% total weight of the matrix. A first-combination of phoenoxyethanol, caprynyl glycol and, sorbic acid ranging from 1.08% to 1.32% of total weight of the matrix is included. Further, a second-combination is provided of oryza sativa bran extract, rosmarinus officinalis leaf extract, helianthus anuus extract and, tocopherol ranging from 0.9% to 1.1% of total weight of the matrix functional as a suitable the topical pain relief composition.

### CROSS REFERENCE TO RELATED APPLICATION

The present application is related to and claims priority to U.S. Provisional Patent Application No. 62/610,899 filed Dec. 27, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of topical compositions and more specifically relates to pain relief compositions.

### 2. Description of Related Art

Pain in the joints or muscles can interfere with daily activities, such as walking, standing, driving, lifting objects, and more. Without an effective way to manage or eliminate the pain, an individual's quality of life may suffer. A suitable solution is desired.

U.S. Pat. No. 7,282,224 to Joy E. Roederer relates to a pain relief composition. The described pain relief composition includes a pain relief composition comprising an effective amount of a nerve inhibiting component, including capsaicin, a capsaicinoid or a capsaicin analogue, which numbs or inhibits the nerve endings that signal pain, in combination with at least one of the following: an effective amount of an inflammation control component which is designed to reduce immediate pain and discourage future pain in the joints and muscles; an effective amount of a cooling component; an effective amount of a heat minimizing or blocking component; an effective amount of a circulation increasing component which effectuates better penetration of the actives to the skin and nerves; and an effective amount of a soothing and anti-inflammatory complex for the joints and/or muscles comprising Glucosamine sulfate or HCl, Zingiber officiniale (Ginger Root) extract, Methyl sulfonylmethane (MSM), Polygonum cuspidatum (Mexican Bamboo) extract, Aloe barbadensis leaf, and Salix alba (white willow) bark extract. Additionally, the composition includes an encapsulation or entrapment system for a timed release delivery.

### BRIEF SUMMARY OF THE INVENTION

In view of the foregoing disadvantages inherent in the known topical compositions art, the present disclosure provides a novel topical pain relief composition. The general purpose of the present disclosure, which will be described subsequently in greater detail, is to provide a topical analgesic in the form of a cream which may be used for a user with pain in joints or muscles.

A topical pain relief composition is disclosed herein. The topical pain relief composition comprises a matrix including a water soluble active ingredient ranging from 20.7% to 25.2% total weight of the matrix, a water insoluble active ingredient ranging from 18.7% to 22.9% total weight of the matrix, and purified water ranging from 56% to 68.8% total weight of the matrix. A first-combination of phoenoxyethanol, caprynyl glycol and, sorbic acid ranging from 1.08% to 1.32% of total weight of the matrix is included. Further, a second-combination is provided of oryza sativa bran extract, rosmarinus officinalis leaf extract, helianthus anuus extract and, tocopherol ranging from 0.9% to 1.1% of total weight of the matrix functional as a suitable the topical pain relief composition.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a topical pain relief composition, constructed and operative according to the teachings of the present disclosure.

FIG. 1 is a perspective view of the topical pain relief composition during an ‘in-use’ condition, according to an embodiment of the disclosure.

FIG. 2 is a perspective view of the topical pain relief composition of FIG. 1, according to an embodiment of the present disclosure.

FIG. 3 is an ingredient list of the topical pain relief composition of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a topical composition and more particularly to a topical pain relief composition as used to improve the effective use of the disclosed pain relief composition.

Generally, the present invention provides a topical cream that may be rubbed on any area in need of pain relief. It offers an analgesic formulated for relieving pain in the joints or muscles. The cream eliminates or reduces pain so that individuals can perform daily activities and routines. The present invention improves an individual's quality of life by helping them manage pain and discomfort. MIRAFLEX assists in relieving pain and discomfort in the joints and muscles. The invention is comprised of a topical analgesic in the form of a cream. The cream is packaged in a five-layer aluminum foil lined tube that holds 4 ounces of cream and includes an open peel seal with a flip top lid.

The ingredients of the cream include water, carbomer, aloe barbadensis leaf extract, dimethyl sulfone, pentylene glycol, glyceryl stearate, cetearyl alcohol, stearyl alcohol, methyl salicylate, cyclomethicone, broccoli seed oil, ultra emu oil, trithanolamine, rice bran extract, rosemary leaf extract, sunflower extract, tocopherol, phoenoxyethanol, caprynyl glycol, sorbic acid, willow bark extract, capsicum frutesdens extract, rice callus cuture extract, menthol, and SD alcohol 40. Sister product (alternate embodiment of the product) will be called/title “MiraFlex-Pro”. Pro contains CBD oil to the already proven Miraflex-R3 cream. This analgesic cream quickly stops pain and calms inflammation, the root cause of chronic pain. Researchers suggest that the non-psychoactive compounds in marijuana, such as CBD, could be a new treatment for chronic pain. CBD is already in use for some conditions that cause chronic pain, such as multiple sclerosis and fibromyalgia.

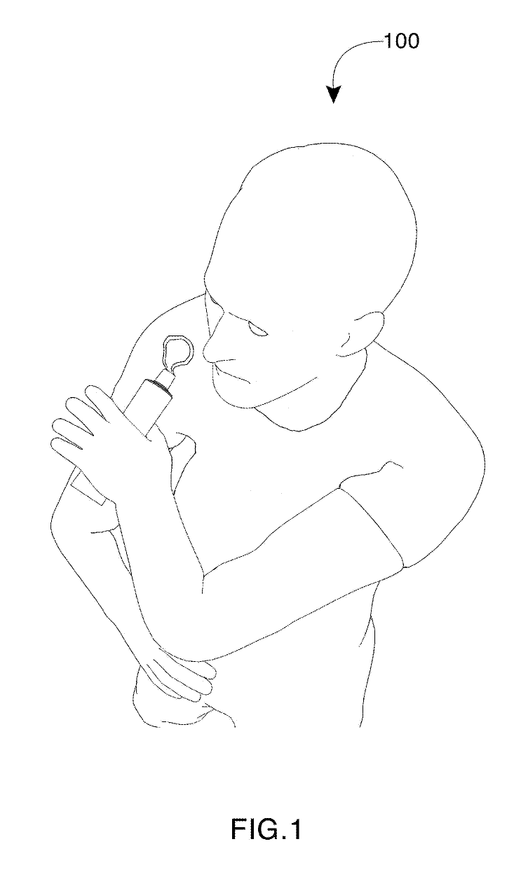

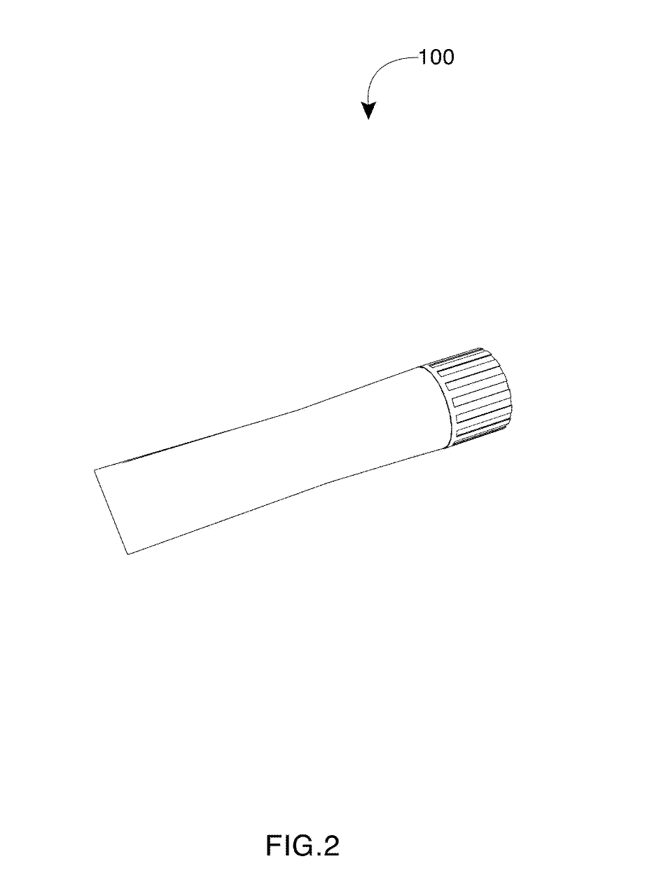

Referring now more specifically to the drawings by numerals of reference, there is shown in FIGS. 1-2, various views of a topical pain relief composition 100. FIGS. 1-2 show a topical pain relief composition 100 according to an embodiment of the present disclosure. As illustrated, the topical pain relief composition 100 comprises a matrix including a water soluble active ingredient ranging from 20.7% to 25.2% total weight of the matrix, a water insoluble active ingredient ranging from 18.7% to 22.9% total weight of the matrix, and purified water ranging from 56% to 68.8% total weight of the matrix. A first-combination is including comprising phoenoxyethanol, caprynyl glycol and, sorbic acid ranging from 1.08% to 1.32% of total weight of the matrix. A second-combination is provided of oryza sativa bran extract, rosmarinus officinalis leaf extract, helianthus anuus extract and, tocopherol ranging from 0.9% to 1.1% of total weight of the matrix functional as a suitable the topical pain relief composition. The composition provides a topical analgesic in the form of a cream which may be used for a user with pain in joints or muscles. It may be rubbed on any area with pain for pain relief. The present invention solves pain so that users can function in their daily lives and routines.

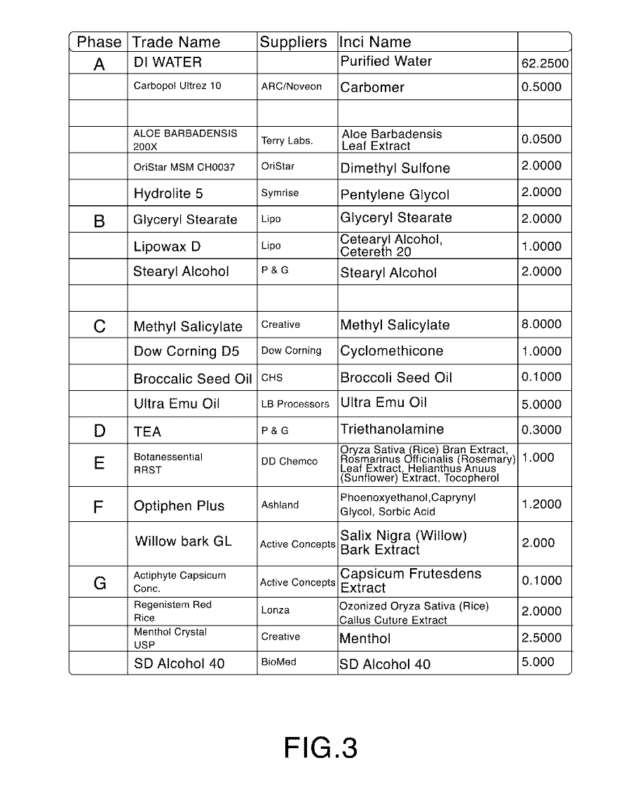

FIG. 3 shows an ingredient list of the topical pain relief composition 100 of FIG. 1, according to an embodiment of the present disclosure. As above, the topical pain relief composition 100 may include a matrix including a water soluble active ingredient, a water insoluble active ingredient, and purified water. In a preferred embodiment, the water soluble active ingredient includes a carbomer ranging from 0.45% to 0.55% by total weight of the matrix. The water soluble active ingredient includes an aloe barbadensis leaf extract ranging from 0.045% to 0.055% by total weight of the matrix. The water soluble active ingredient includes a dimethyl sulfone ranging from 1.8% to 2.2% by total weight of the matrix. The water soluble active ingredient includes a pentylene glycol ranging from 1.8% to 2.2% by total weight of the matrix. The water soluble active ingredient includes a glyceryl stearate ranging from 1.8% to 2.2% by total weight of the matrix. The water soluble active ingredient includes a cetearyl alcohol ranging from 0.9% to 1.1% by total weight of the matrix. The water soluble active ingredient includes a triethanolamine ranging from 0.27% to 0.33% by total weight of the matrix. The water soluble active ingredient includes a salix nigra bark extract ranging from 1.8% to 2.2% by total weight of the matrix. The water soluble active ingredient includes a capsicum frutesdens extract ranging from 0.9% to 1.1% by total weight of the matrix. The water soluble active ingredient may further include an ozonized oryza sativa callus culture extract ranging from 0.9% to 1.1% by total weight of the matrix. The water soluble active ingredient includes a specially denatured alcohol ranging from 4.5% to 5.5% by total weight of the matrix.

As above, the topical pain relief composition 100 may include a matrix including a water soluble active ingredient, a water insoluble active ingredient, and purified water. The water insoluble active ingredient includes a stearyl alcohol ranging from 1.8% to 2.2% by total weight of the matrix. The water insoluble active ingredient includes a methyl salicylate ranging from 7.2% to 8.8% by total weight of the matrix. The water insoluble active ingredient includes a cyclomethicone ranging from 0.9% to 0.11% by total weight of the matrix. Further, the water insoluble active ingredient includes a broccoli seed oil ranging from 0.09% to 0.11% by total weight of the matrix. The water insoluble active ingredient includes a menthol ranging from 2.25% to 2.75% by total weight of the matrix.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A topical pain relief composition comprising:
a matrix including:
a water soluble active ingredient ranging from 20.7% to 25.2% total weight of the matrix;
a water insoluble active ingredient ranging from 18.7% to 22.9% total weight of the matrix;
purified water ranging from 56% to 68.8% total weight of the matrix;
a first-combination of: phoenoxyethanol, caprynyl glycol and, sorbic acid ranging from 1.08% to 1.32% of total weight of the matrix; and
a second-combination of: oryza sativa bran extract, rosmarinus officinalis leaf extract, helianthus anuus extract and, tocopherol ranging from 0.9% to 1.1% of total weight of the matrix functional as a suitable said topical pain relief composition.

2. The topical pain relief composition of claim 1, wherein said water soluble active ingredient includes a carbomer ranging from 0.45% to 0.55% by total weight of the matrix.

3. The topical pain relief composition of claim 1, wherein said water soluble active ingredient includes an aloe barbadensis leaf extract ranging from 0.045% to 0.055% by total weight of the matrix.

4. The topical pain relief composition of claim 1, wherein said water soluble active ingredient includes a dimethyl sulfone ranging from 1.8% to 2.2% by total weight of the matrix.

5. The topical pain relief composition of claim 1, wherein said water soluble active ingredient includes a pentylene glycol ranging from 1.8% to 2.2% by total weight of the matrix.

6. The topical pain relief composition of claim 1, wherein said water soluble active ingredient includes a glyceryl stearate ranging from 1.8% to 2.2% by total weight of the matrix.

7. The topical pain relief composition of claim 1, wherein said water soluble active ingredient includes a cetearyl alcohol ranging from 0.9% to 1.1% by total weight of the matrix.

8. The topical pain relief composition of claim 1, wherein said water soluble active ingredient includes a triethanolamine ranging from 0.27% to 0.33% by total weight of the matrix.

9. The topical pain relief composition of claim 1, wherein said water soluble active ingredient includes a salix nigra bark extract ranging from 1.8% to 2.2% by total weight of the matrix.

10. The topical pain relief composition of claim 1, wherein said water soluble active ingredient includes a capsicum frutesdens extract ranging from 0.9% to 1.1% by total weight of the matrix.

11. The topical pain relief composition of claim 1, wherein said water soluble active ingredient includes an ozonized oryza sativa callus culture extract ranging from 0.9% to 1.1% by total weight of the matrix.

12. The topical pain relief composition of claim 1, wherein said water soluble active ingredient includes a specially denatured alcohol ranging from 4.5% to 5.5% by total weight of the matrix.

13. The topical pain relief composition of claim 1, wherein said water insoluble active ingredient includes a stearyl alcohol ranging from 1.8% to 2.2% by total weight of the matrix.

14. The topical pain relief composition of claim 1, wherein said water insoluble active ingredient includes a methyl salicylate ranging from 7.2% to 8.8% by total weight of the matrix.

15. The topical pain relief composition of claim 1, wherein said water insoluble active ingredient includes a cyclomethicone ranging from 0.9% to 0.11% by total weight of the matrix.

16. The topical pain relief composition of claim 1, wherein said water insoluble active ingredient includes a broccoli seed oil ranging from 0.09% to 0.11% by total weight of the matrix.

17. The topical pain relief composition of claim 1, wherein said water insoluble active ingredient includes a menthol ranging from 2.25% to 2.75% by total weight of the matrix.

18. A topical pain relief composition, the topical pain relief composition comprising:
a matrix including:
a water soluble active ingredient ranging from 20.7% to 25.2% total weight of the matrix;
a water insoluble active ingredient ranging from 18.7% to 22.9% total weight of the matrix;
purified water ranging from 56% to 68.8% total weight of the matrix;
a first-combination of: phoenoxyethanol, caprynyl glycol and, sorbic acid ranging from 1.08% to 1.32% of total weight of the matrix;
a second-combination of: oryza sativa bran extract, rosmarinus officinalis leaf extract, helianthus anuus extract and, tocopherol ranging from 0.9% to 1.1% of total weight of the matrix functional as a suitable said topical pain relief composition;
wherein said water soluble active ingredient includes a carbomer ranging from 0.45% to 0.55% by total weight of the matrix;
wherein said water soluble active ingredient includes an aloe barbadensis leaf extract ranging from 0.045% to 0.055% by total weight of the matrix;
wherein said water soluble active ingredient includes a dimethyl sulfone ranging from 1.8% to 2.2% by total weight of the matrix;
wherein said water soluble active ingredient includes a pentylene glycol ranging from 1.8% to 2.2% by total weight of the matrix;
wherein said water soluble active ingredient includes a glyceryl stearate ranging from 1.8% to 2.2% by total weight of the matrix;
wherein said water soluble active ingredient includes a cetearyl alcohol ranging from 0.9% to 1.1% by total weight of the matrix;
wherein said water soluble active ingredient includes a triethanolamine ranging from 0.27% to 0.33% by total weight of the matrix;
wherein said water soluble active ingredient includes a salix nigra bark extract ranging from 1.8% to 2.2% by total weight of the matrix;
wherein said water soluble active ingredient includes a capsicum frutesdens extract ranging from 0.9% to 1.1% by total weight of the matrix;
wherein said water soluble active ingredient includes an ozonized oryza sativa callus culture extract ranging from 0.9% to 1.1% by total weight of the matrix;
wherein said water soluble active ingredient includes a specially denatured alcohol ranging from 4.5% to 5.5% by total weight of the matrix;
wherein said water insoluble active ingredient includes a stearyl alcohol ranging from 1.8% to 2.2% by total weight of the matrix;
wherein said water insoluble active ingredient includes a methyl salicylate ranging from 7.2% to 8.8% by total weight of the matrix;
wherein said water insoluble active ingredient includes a cyclomethicone ranging from 0.9% to 0.11% by total weight of the matrix;
wherein said water insoluble active ingredient includes a broccoli seed oil ranging from 0.09% to 0.11% by total weight of the matrix; and
wherein said water insoluble active ingredient includes a menthol ranging from 2.25% to 2.75% by total weight of the matrix.
